(19) United States
(12) Patent Application Publication
(10) Pub. No.: US 2021/0393448 A1
(43) Pub. Date: Dec. 23, 2021

(54) METHODS AND APPARATUS FOR MONITORING PRESENCE OF URINE OR FECES

(71) Applicant: MAVIN WEAR INC., San Jose, CA (US)

(72) Inventors: Lu WANG, San Jose, CA (US); JiChao SUN, San Jose, CA (US)

(21) Appl. No.: 17/292,514

(22) PCT Filed: Nov. 8, 2019
(86) PCT No.: PCT/US2019/060588

§ 371 Date: May 10, 2021

Related U.S. Application Data
(60) Provisional application No. 62/757,947, filed on Nov. 9, 2018.

(51) Int. Cl. A61F 13/42; G06K 7/10; G06K 19/07; G01N 27/02; G01N 33/487; G01N 33/483; G01N 33/493

(52) U.S. Cl. CPC A61F 13/42; G06K 7/10366; G06K 19/0723; A61F 2013/424; G01N 33/48707; G01N 33/4833; G01N 33/493; G01N 27/02

(57) ABSTRACT

Discloses herein are systems and methods for determining waste, such as urine or feces. Also disclosed herein are diapers incorporating a system as described herein for detecting waste, such as urine or feces, when worn by a subject.

### CROSS-REFERENCE

This application claims the benefit of U.S. Provisional Patent Application No. 62/757,947, filed on Nov. 9, 2018, which is hereby incorporated by reference in its entirety.

### SUMMARY

Disclosed herein are systems for detecting waste (e.g. urine or feces). A system can comprise a processor, a battery, and at least one sensor. A battery can comprise an anode and a cathode. A battery may not be active in the absence of urine, feces, or an electrolyte solution. A battery may be activated when contacted with urine, feces, or an electrolyte solution; thereby providing power to the processor and sensor in the presence of urine, feces, or an electrolyte solution. In some cases, an electrical circuit (for example, comprising a battery) can be substantially open in the absence of urine, feces, or an electrolyte solution. In some cases, an electrical circuit can be at least partially closed when a sensor is contacted with urine, feces, or an electrolyte solution. A sensor can comprise a working electrode and a counter electrode. A sensor can be operatively coupled to a battery and a processor. A processor can be configured to measure an impedance across a working electrode and a counter electrode of at least one sensor in response to feces or urine, thereby generating data that can be indicative of urine, feces, or a combination thereof. In some embodiments, a system can further comprise a radio-frequency identification (RFID) transponder operatively coupled to a processor and a battery. In some embodiments, a RFID transponder can be configured to transmit data when a battery is activated. In some embodiments, a system may not be active in the absence of urine or feces. In some embodiments, urine, feces, or a combination thereof can activate a system. In some embodiments, data can be impedance data. In some embodiments, a system can comprise a memory module operatively coupled to a processor and a battery, where a memory module can be configured to store the data. In some embodiments, an RFID transponder can be configured to access the memory module. In some embodiments, a system can further comprise an RFID receiver operatively coupled to a processor and a battery. In some embodiments, an RFID receiver can be configured to receive instructions from a device by an RF signal to transmit data to the device. In some embodiments, an RFID transponder can be configured to transmit data to a device by an RF signal. In some embodiments, a device can be a mobile device. In some embodiments, generating data can comprise an analog to digital conversion of a voltage. In some embodiments, a system can comprise a plurality of sensors. In some embodiments, a plurality of sensors can be arranged in at least two layers on a surface. In some embodiments, a sensor can comprise a glucose sensor.

Also disclosed herein are diapers that can comprise a system as described herein and an absorbent pad. In some embodiments, diaper can comprise a reservoir storing an electrolyte solution. In some embodiments, a reservoir can comprise a frangible membrane. In some embodiments, an electrolyte solution can be at least partially sequestered from a battery by a frangible membrane. In some embodiments, an electrolyte solution can contact a battery when a frangible membrane is ruptured. In some embodiments, a rupturing of a frangible membrane can activate a battery. In some embodiments, a transmitter can be configured to transmit impedance data to a device. In some embodiments, a battery can comprise magnesium or a metal thereof. In some embodiments, a battery can comprise from about 10% to about 90% by weight of a magnesium or metal thereof with respect to a total weight of a battery. In some embodiments, a battery can comprise copper or a metal thereof. In some embodiments, a battery can comprise from about 10% to about 90% by weight of a copper or metal thereof with respect to a total weight of a battery. In some embodiments, an object can be configured to pierce a frangible membrane, thereby contacting an electrolyte with a battery. In some embodiments, an absorbent pad can comprise a plurality of sensors. In some embodiments, a plurality of sensors can be arranged in at least two layers on an absorbent pad. In some embodiments, a diaper can further comprise a porous or hydrophobic material layer covering a plurality of sensors. In some embodiments, a diaper can further comprise a visual indicator configured to provide a visual prompt in the presence of urine, feces, or a combination thereof. In some embodiments, a diaper can further comprise an audio indicator configured to provide an audio prompt in the presence of urine, feces, or a combination thereof. In some embodiments, a diaper can further comprise a processor operatively coupled to at least one sensor and a battery. In some embodiments, a processor is configured to measure an impedance across a working electrode and a counter electrode of at least one sensor in response to feces or urine. In some embodiments, the urine or feces can be in contact with an absorbent pad, thereby generating impedance data that can be indicative of urine or feces. In some embodiments, a diaper can be sized to fit a human. In some embodiments, a human can be from about 0 years old to about 17 years old. In some embodiments, a human can be from about 0 years old to about 4 years old. In some embodiments, a human can be from about 18 years old to about 120 years old. In some embodiments, a human can be from about 65 years old to about 120 years old.

Also disclosed herein are methods for detecting urine or feces. A method can comprise detecting feces or urine by contacting feces or urine with at least one sensor of a system described herein; thereby generating data; and transmitting data to a device using an RFID transponder. In some embodiments, data can be stored on a mobile device or a stationary device. In some embodiments, analysis of data can be performed on a mobile device or a stationary device. In some embodiments, analysis can comprise an alert of a presence of urine or feces. In some embodiments, analysis can comprise measuring a presence of urine or feces as a function of time. In some embodiments, a method can further comprise rupturing a frangible membrane, thereby contacting a battery with an electrolyte solution. In some embodiments, a detecting can be performed on a human. In some embodiments, a human can be from about 0 years old to about 17 years old. In some embodiments, a human can be from about 0 years old to about 4 years old. In some embodiments, a human can be from about 18 years old to about 120 years old. In some embodiments, a human can be from about 65 years old to about 120 years old.

Also disclosed herein are methods for detecting urine or feces that can comprise detecting feces or urine by contacting feces or urine with an absorbent pad of a diaper described herein; thereby generating data; and transmitting data to a device using a transmitter. In some embodiments, data can be stored on a mobile device or a stationary device. In some embodiments, data can be uploaded to a remote server via a cloud based system. In some embodiments, analysis of data can be performed on a mobile device or a stationary device. In some embodiments, analysis of data can be performed on a remote server. In some embodiments, analysis can comprise an alert of a presence of urine or feces. In some embodiments, analysis can comprise an alert of urine saturation based on a duration the urine or a frequency of data. In some embodiments, analysis can comprise measuring a presence of urine or feces as a function of time. In some embodiments, a method can further comprise rupturing a frangible membrane, thereby contacting a battery with an electrolyte solution. In some embodiments, a detecting can be performed on a human. In some embodiments, a human can be age 0-17 years old. In some embodiments, a human can be age 18-120 years old. In some embodiments, a sensor can comprise a glucose sensor. In some cases, a method can further comprise detecting a level of glucose in the urine, feces, or combination thereof by contacting urine, feces, or a combination thereof with a glucose sensor. In some embodiments, a method can further comprise monitoring a level of glucose in the urine, feces, or combination thereof over a period of time. In some embodiments, a method can further comprise determining compliance with a diabetic treatment based on a level of glucose in a subject administered a diabetic treatment. In some embodiments, a method can further comprise administering a medicament to a human. In some embodiments, a medicament can be selected from the group consisting of: diaper rash cream, zinc oxide, petrolatum, hyaluronic acid, aloe vera, shea butter, jojoba, coconut oil, calendula, vitamin B5, vitamin E, BPH treatment, diarrhea treatment, loperamide, diabetic treatment, insulin, and any combination thereof.

### BRIEF DESCRIPTION OF THE DRAWINGS

The novel features of exemplary embodiments are set forth with particularity in the appended claims. A better understanding of the features and advantages will be obtained by reference to the following detailed description that sets forth illustrative embodiments, in which the principles of exemplary embodiments are utilized, and the accompanying drawings of which:

FIG. 1 illustrates an example of the system that detects the presence and amount of urine and feces.

FIG. 2 illustrates an example of how a single cell in a disposable battery is constructed.

FIG. 3 illustrates an example of how one or more cells can be connected to form a disposable battery.

FIG. 4 illustrates an example of the pattern of electrodes used to detect urine and feces.

FIG. 5 illustrates an example of material that covers the electrodes

FIG. 6 illustrates an example of an electrolyte chamber separated from a battery cell by a frangible membrane

### DETAILED DESCRIPTION

Overview

Disclosed herein are systems for detection of urine and feces. A system can detect a presence of urine or feces, can estimate an amount of urine or feces, and can capture a frequency of urine or feces over a period of time. Such detection can be communicated to a device. In particular, disclosed herein are methods and materials for constructing a low-cost, non-toxic, and fully disposable system with powering, sensing, and data transmitting modules.

Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” can mean within an acceptable error range for the particular value as determined by one of ordinary skill in the art, which will depend in part on how the value is measured or determined, e.g., the limitations of the measurement system. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed. Also, where ranges and/or subranges of values are provided, the ranges and/or subranges can include the endpoints of the ranges and/or subranges.

The term “substantially” as used herein can refer to a value approaching 100% of a given value. In some cases, the term can refer to an amount that can be at least about 90%, 91%, 92%, 93%, 94%, 95%, 96%, 97%, 98%, 99%, 99.9%, or 99.99% of a total amount. In some cases, the term can refer to an amount that can be about 100% of a total amount.

The term “subject”, “patient” or “individual” as used herein can encompass a mammal and a non-mammal. A mammal can be any member of the Mammalian class, including but not limited to a human, a non-human primates such as a chimpanzee, an ape or other monkey species; a farm animal such as cattle, a horse, a sheep, a goat, a swine; a domestic animal such as a rabbit, a dog (or a canine), and a cat (or a feline); a laboratory animal including a rodent, such as a rat, a mouse and a guinea pig, and the like. A non-mammal can include a bird, a fish and the like. In some embodiments, a subject can be a mammal. In some embodiments, a subject can be a human. In some instances, a human can be an adult. In some instances, a human can be a child. In some instances, a human can be age 0-17 years old. In some instances, a human can be age 0-4 years old.

In some instances, a human can be age 18-120 years old. In some instances, a human can be age 65-120 years old. In some instances, a subject can be a male. In some instances, a subject can be a female. In some instances, a subject can be diagnosed with, or can be suspected of having, a condition or disease. A subject can be a patient. A subject can be an individual. In some instances, a subject, patient or individual can be used interchangeably.

System

Disclosed herein are systems for detection of waste. In some cases, waste can be urine, feces, or a combination of urine and feces.

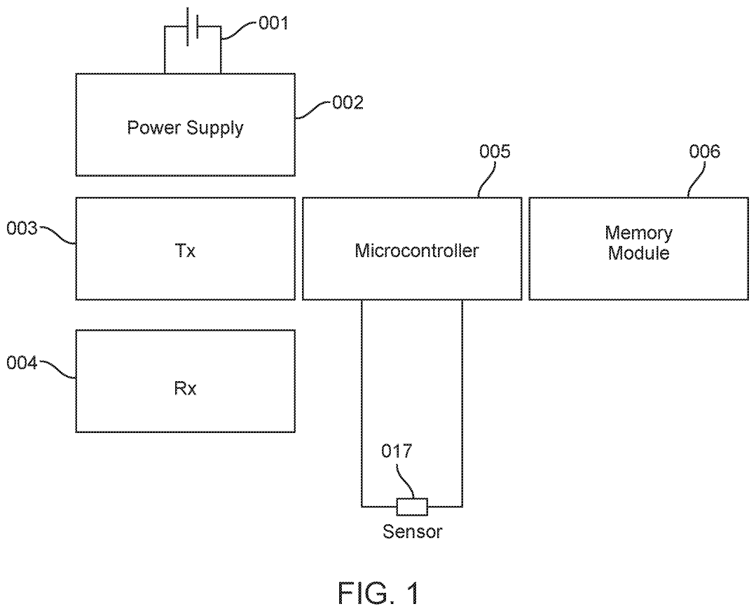

FIG. 1 depicts a schematic of an exemplary embodiment of a system described herein. As depicted in FIG. 1, a system can include a battery 001, a power supply module 002 that can supply a stable voltage, a transmitter 003, a receiver 004, a controller 005, a memory module 006 and a sensor 017. Battery 001 and power supply 002 can supply a voltage to all system components.

A system can comprise a controller 005. In some cases, a controller 005 can be a microprocessor. A microprocessor can be a CPU as a discrete component. In some cases, a controller 005 can be a microcontroller. A microcontroller can contain an integrated CPU processor core, computer memory, program memory, and/or a programmable input/output peripheral. In some cases, program memory can include ferroelectric RAM, NOR flash, OTP ROM, or RAM. A controller 005 can supply an AC voltage to sensor 017 through a D/A converter. A control module 005 can read a current to sensor 017 through an A/D converter.

Voltage can be applied across sensor 017 for detection. A voltage can be applied using a pre-programmed or a fixed current or voltage to calculate admittance, impedance, conductance, resistance, or voltage, and/or detect changes to these parameters in the presence of conductive waste material such as feces or urine. For example, because the presence of feces can create a lower resistance or impedance path, and a higher admittance path between electrodes of sensor 017, increasing amounts of feces can cause lower resistance or impedance paths and a higher admittance between electrodes, lowering the net impedance or resistance between the electrodes and raising net admittance in the electrode array. In some cases, electrodes can comprise opposed finger electrodes. In some cases, electrodes can comprise staggered parallel electrodes. In some cases, electrodes can comprise zigzagging parallel electrodes.

Applied current or voltage can be either alternating (AC) or direct (DC). Applied voltages may be from about 0.000V to 0.005V RMS, from about 0.005V to about 0.05V RMS, from about 0.05V to about 0.5V RMS, or from about 5V to about 50V RMS. A frequency of applied voltage may be from about 0.1 Hz to about 1 Hz, from about 10 Hz to about 100 Hz, from about 100 Hz to about 1,000 Hz, from about 1,000 Hz to about 10,000 Hz, from about 10,000 Hz to about 100,000 Hz, or from about 100,000 Hz to about 1,000,000 Hz. In some cases, a resistor, capacitor, inductor, or other electrical component can comprise all or a portion of sensor 017 circuitry. For example, a resistor, capacitor, or inductor can be connected in parallel or in series with the electrode arrays to determine whether the electrode arrays are electrically open or closed.

Voltage or other values can be converted using an analog-to-digital convertor (ADC), and in some examples an algorithm can be executed by one or more processors to process data and determine the presence of feces or urine.

In some cases, a system can comprise a full-analog circuit. In some cases, a full analog circuit can be used for impedance or admittance calculation. In some cases, a full analog circuit can reduce usage of battery power and extend battery life. In some cases, RF transmission can be accomplished in an analog circuit. In some cases, a customized analog circuit can perform calculation, data transmission or a combination of these functions. In some cases, a digital circuit can perform calculation, data transmission or a combination of these functions. In some cases, a circuit can comprise bare die. In some cases, a circuit can be flexible. In some cases, a circuit can be incorporated into a flexible package.

In some cases, impedance or admittance can be calculated across a sensor 017. Impedance or admittance can be calculated by controller 005 and stored in memory module 006. Receiver 004 can receive a signal to transfer data to a device. A receiver 004 can transmit all or partial data (e.g. impedance or admittance data) stored in memory module 006.

A transmitter 003 can transmit data (e.g. impedance, resistance, admittance, conductance, capacitance, etc.) to a receiving device (e.g. a mobile device or a stationary device). A transmitter 003 can transmit data via a wired or a wireless connection. In some cases, a wireless connection can be a Wi-Fi, RF, 3G, 4G LTE, or Bluetooth connection. In some cases, a wired connection can be an Ethernet, fiber optic, coaxial, or USB connection. In some cases, a Bluetooth connection can comprise multiple standards.

A receiver 004 can receive data (e.g. impedance, resistance, admittance, conductance, capacitance, etc.) from a receiving device (e.g. a mobile device or a stationary device). A receiver 004 can receive data via a wired or a wireless connection. In some cases, a wireless connection can be a Wi-Fi, RF, 3G, 4G LTE, or Bluetooth connection. In some cases, a wired connection can be an Ethernet, fiber optic, coaxial, or USB connection.

A system can further comprise additional means for signaling that urine or feces is present. For example, a system can have audible means operatively coupled to the controller 005 that emit an audible signal when urine or feces is present. A system can also include visual signaling means such as a light operatively coupled to the controller 005 that can activate in the presence or urine or feces.

Battery

A battery 001 can provide power to system components as described herein. A battery 001 can comprise an anode and a cathode. An anode or a cathode can contain a conductive metal or a salt thereof. Examples of metals can include zinc, carbon (graphite), manganese, nickel, lithium, mercury, silver, cadmium, and lead. Examples of metal salts can include zinc chloride, zinc dioxide, manganese dioxide, cupric oxide, iron (II) disulfide, lithium chromate, lithium chromite, and mercuric oxide. Exemplary batteries can include zinc-carbon, zinc-chloride, zinc-manganese dioxide, nickel oxyhydroxide, lithium-cupric oxide, lithium-iron (II) disulfide, lithium-chromite, mercury oxide, zinc-air, silver-oxide, nickel-cadmium, lead-sulfuric acid, nickel-zinc, copper-magnesium, silver-zinc, and lithium ion batteries. In some cases, zinc-air batteries can comprise a membrane. In some cases, a membrane can cover one or more air holes.

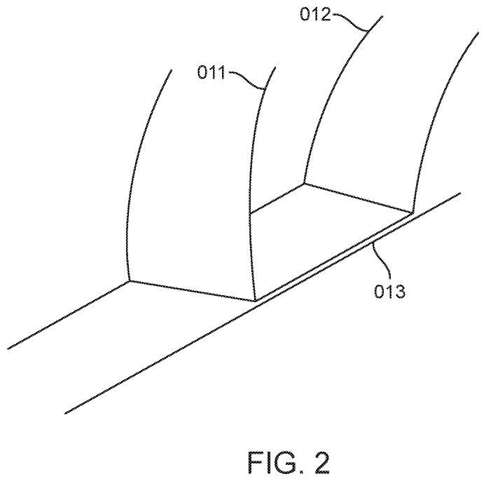

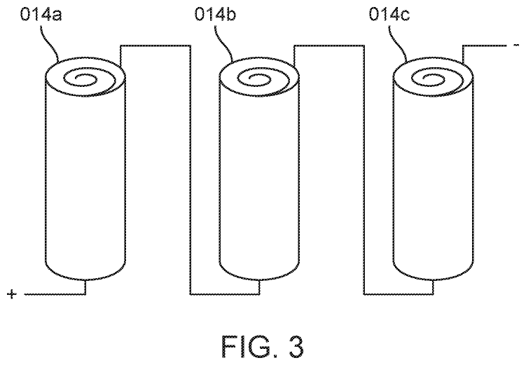

FIG. 2 depicts an exemplary battery design that uses magnesium and copper sheets as anode 012 and cathode 013. A porous, hydrophilic, non-conductive material 011 can be used to insulate anode 012 and cathode 013. In some cases, a non-conductive material 011 can absorb electrolyte. In some cases, a non-conductive material 011, an anode 012, and a cathode 013 can be rolled into a cylinder. In some cases, a battery 001 can comprise multiple battery cells connected in serial or parallel. FIG. 3 depicts a battery with battery cells 014A, 014B, and 014C connected in series to provide a higher output voltage. A battery 001 can comprise at least about 1, 2, 3, 4, 5, 6, 7, 8, 9, or more than 10 battery cells connected in series or parallel. In some cases, a cell can comprise a plurality of electrodes. In some cases, a plurality of electrodes can comprise about 2, about 3, about 4, about 5, about 6, about 7, about 8, about 9, about 10, or more than about 10 electrodes. In some cases, electrodes can comprise a disc shape. In some cases, a disc shape can maximize electrode surface area.

In some cases, each cell in a battery 001 can contain a vacuum space, such that when gas bubbles are formed on the electrode surface, it is suctioned into the vacuum space instead of sticking to the electrode surface. The removal of bubbles can increase a battery's maximum output current, as the presence of bubbles can cause reduction in electrode surface area.

A battery 001 can be in an active or an inactive configuration prior to use. In some cases, a battery 001 can be inactive. In some cases, in an inactive configuration, an anode 012 and a cathode 013 may have no electrolyte in a cell. In some cases, an inactive configuration can comprise a configuration in which an anode 012 and a cathode 013 are at least partially insulated from each other by material with a high resistance. In some cases, while inactive a battery may comprise part of a circuit that is substantially open. In some cases, while inactive, a battery 001 may not provide power to system components. In some cases, an inactive battery can become active when a circuit is at least partially closed. In some cases, a circuit can become at least partially closed when an insulating material between an anode and a cathode are contacted with a substance that can act as a conductor. In some cases, when a material between an anode and a cathode are contacted with a substance that can act as a conductor an electrical impedance of a material can decrease. An electrolyte solution can also be selected to increase a voltage and/or current output through a system by varying the ionic strength of the electrolyte solution. For example, an electrolyte solution with an increased ionic strength can increase the reactivity between the electrolyte in the electrolyte solution and the anode. Accordingly, increased or decreased ionic strength electrolyte solutions can be used to module the voltage and/or current output by modulating the activity at the anode. In some cases, when a material between an anode and a cathode are contacted with a substance that can act as a conductor, the electrical admittance of a material can increase. In some cases, an increase in admittance can at least partially close an electrical circuit. In some cases, at least partially closing an electrical circuit can cause a current to flow through an electrical circuit. In some cases, a current flowing through a circuit can be a result of a battery being active. In some cases, a substance that can activate a battery 001 can include urine, feces, an electrolyte, glucose, gluconic acid, and any combination thereof.

In an embodiment, an electrolyte can be used to activate a battery. An electrolyte may be acidic, neutral or basic. Examples of acidic electrolytes can include sulfuric acid, nitric acid, chloric acid, phosphoric acid, citric acid, hydrochloric acid, citric acid, and acetic acid. Examples of basic electrolytes can include potassium hydroxide, sodium hydroxide, sodium acetate, sodium bicarbonate, an imidazolium salt, a pyrrolidinium salt, a piperidinium salt, an ammonium salt, urea, and N-methyl-N-propylpiperidinium-bis(trifluoromethanesulfonyl)imide. Examples of neutral electrolytes can include water, sodium chloride, copper chloride, lithium chloride, aluminum chloride, potassium chloride, lithium hexafluorophosphate, sodium hexafluorophosphate, potassium hexafluorophosphate, sodium perchlorate, and sodium difluoro(oxalate)borate.

In an embodiment, the electrolyte may be urine or feces. Urine or feces may act as an electrolyte, which can activate a battery 001 by bridging the insulated anode 012 and cathode 013. In some instances lacking an exogenous electrolyte solution, a battery 001 may be active only in the presence of urine or feces.

Applications

A system as described herein can be incorporated into a diaper or other incontinence product. A diaper comprising a system as described herein can be used to monitor the presence of urine or feces in a subject wearing the diaper. A diaper can sized to fit a human infant, toddler, or child. In some cases, a diaper can sized to fit a human adult. In addition to the signaling means described herein, a diaper can further comprise a color change stripe that reacts to moisture, which can provide additional indication of the presence of urine or feces

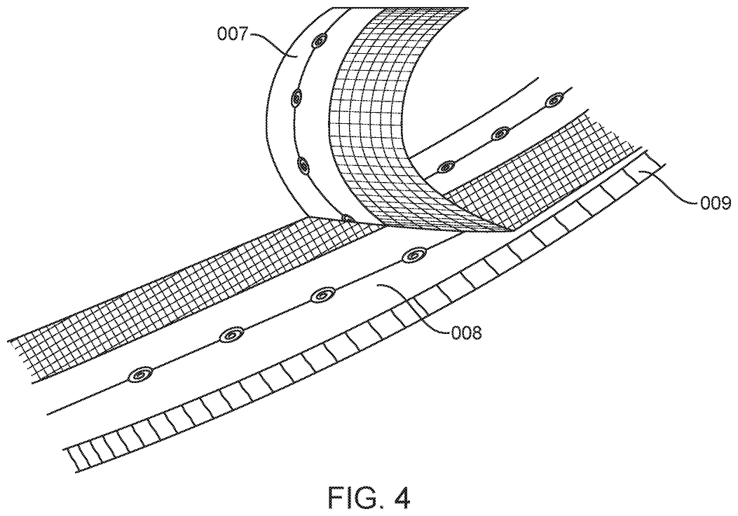

A system as described herein can be incorporated into a diaper to allow for detection of urine or feces. For instance, a sensor 017 as described herein can be placed in an absorbent pad of a diaper. Sensor 017 can have a pair of elongate electrodes. In some cases, a sensor can comprise a conductive ink. In some cases, a conductive ink can comprise a polymer. In some cases, a polymer can comprise a carbon-doped polymer. In some cases, a polymer can comprise a metal-doped polymer. In some cases, a polymer can be in an organic solvent. In some cases, a conductive ink can be dispensed by a syringe to a surface. In some cases, a conductive ink can be dispensed by an ink-jet nozzle to a surface. In some cases, a surface can comprise plastic. In some cases, a sensor can comprise a glucose sensor. In some cases, a glucose sensor can comprise at least two layers. In some cases, a first layer can comprise one or more electrodes. In some cases, a second layer can comprise glucose oxidase, ferricyanide or a combination thereof. In some cases, a presence of urine or feces can be detected by the detection of glucose in urine or feces. In some cases, glucose oxidase catalyzes a reaction between glucose and oxygen. In some cases, a reaction between glucose and oxygen can produce gluconic acid and peroxide. In some cases, gluconic acid can react with ferricyanide in a sensor. In some cases, gluconic acid reacting with ferricyanide can produce ferrocyanide. In some cases, ferrocyanide can be measured by applying a voltage between two or more electrodes. In some cases, two or more electrodes can comprise a working and reference electrode. In some cases, an electrode layer can comprise two electrodes. In some cases, a two-electrode configuration can comprise a working electrode and a reference electrode. In some cases, a working electrode surface can comprise gold, platinum, carbon, a material that inert electrochemically, or any combination thereof. In some cases, a carbon can comprise glassy carbon. In some cases, a reference electrode can comprise silver, silver chloride, or a combination thereof. In some cases, a reference can comprise silver with a silver chloride surface. In some cases, a reference electrode can be larger in surface area than a working electrode. In some cases, a voltage can be applied between two or more electrodes. In some cases, a current can be measured across two or more electrodes. In some cases, a fixed current can be applied across two or more electrodes. In some cases, a voltage can be measured across two or more electrodes. In some cases, a trigger electrode can be used to sense a presence of a liquid. In some cases, a trigger electrode can be triggered by an impedance between two or more electrodes reaching a detection threshold. In some cases, a detection threshold can comprise 1 kohm. In some cases, a trigger electrode can be triggered by an admittance between two or more electrodes reaching a detection threshold. In some cases, a detection threshold can comprise 1 kS. In some cases, a liquid may be constantly monitored. In some cases, a sensor can comprise a capillary. In some cases, a capillary can facilitate a stable flow of liquid to a sensor. In some cases, a layer can comprise a hydrophilic material. In some cases, a hydrophilic material can facilitate liquid pass-through. In some cases, a sensor may comprise a thermistor. In some cases, a thermistor may allow temperature compensation of recorded results. In some cases, a thermistor may detect whether a device is being worn. FIG. 4 depicts an embodiment of an arrangement of elongate electrodes 007 and 008 within absorbent pad 009. In some cases, electrodes 007 and 008 can be electrically insulated from each other in the absence of urine or feces.

When incorporated into absorbent pad 009 in a diaper, a sensor 017 can detect urine or feces when excreted by a subject. In some cases, urine can be detected in a diaper. Upon excretion of urine by a subject into the absorbent pad 009, the urine can produce an electrical path between electrodes 007 and 008, thus lowering impedance and raising admittance between the two electrodes. After urine is fully absorbed by the absorbent pad 009 of the diaper, a thin layer of liquid can remain between electrodes 007 and 008, which can result in a higher impedance and a lower admittance than when urine was initially excreted. In some cases, fecal matter can be detected in a diaper. Fecal matter can produce an electrical path between electrodes 007 and 008, which can produce a persistent, low impedance and high admittance between electrodes 007 and 008. In one embodiment, the two electrodes 007 and 008 may be placed on two layer of material, whereby both layers have holes to allow liquid to pass through. In one embodiment, the two electrodes 007 and 008 may be placed on the same layer, whereas the layer is porous or have holes to allow liquid to pass through.

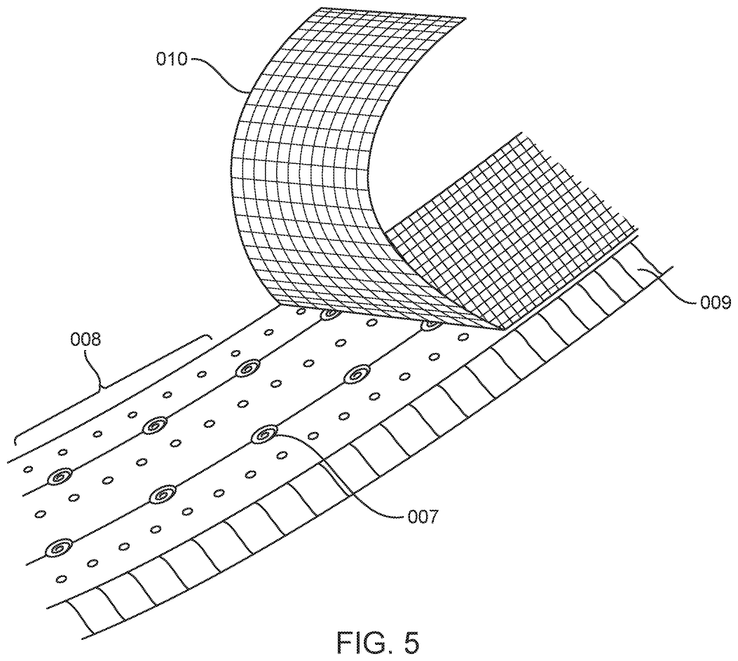

In order to detect urine or feces upon excretion, electrodes 007 and 008 may need to be arranged in a configuration to allow for rapid contact with the excretion. In some embodiments, electrodes 007 and 008 can be placed on the most superficial layer of the diaper absorbent pad. One potential shortcoming is this arrangement is that the electrodes 007 and 008 may be in direct contact with the skin of the subject, which may be wet. Wet skin can produce a low impedance and high admittance electrical path between electrodes 007 and 008, making it difficult to distinguish whether urine or feces are causing the low impedance and high admittance. FIG. 5 depicts an improved configuration. As shown in FIG. 5, a layer of thin, porous or gridded, hydrophobic layer of material 010 can be placed on top of electrodes 007 and 008, which can allow urine to quickly pass through and get absorbed by the absorbent pad 009. In this configuration, direct contact between the skin of the subject and electrodes 007 and 008 can be avoided.

As previously described, a battery 001 can be activated in the presence of urine or feces. In some cases, a battery 001 can be placed in an absorbent pad 009 in a similar manner to electrodes 007 and 008. Thus, when a subject excretes urine or feces into the diaper, the urine or feces can contact absorbent pad 009 and make contact with the battery 001. Activation of battery 001 by urine or feces provides power to the system components. For instances, battery 001 when activated provides power to controller 005. Upon activation, controller 005 measures electrical impedance and admittance at sensor 017, which in the presence of the activating urine or feces may be lower than in the absence of the activating urine or feces. When the controller 005 measures low impedance and high admittance at sensor 017, controller 005 will send a signal to a mobile device or a stationary device via transmitter 003 that urine or feces are present.

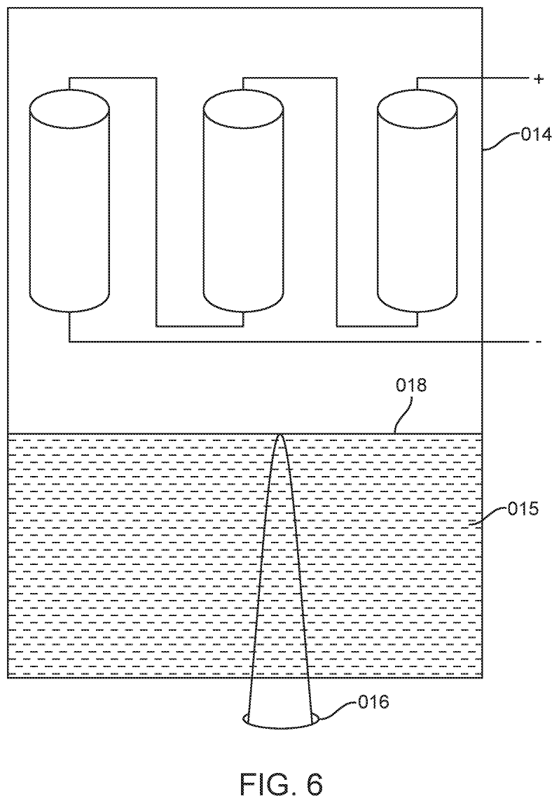

In another embodiment, it is possible to activate the system in the absence of urine or feces using an exogenous electrolyte. An exogenous electrolyte can be stored in a compartment in a diaper and can be physically separated from electrodes 007 and 008 via a frangible membrane 018. FIG. 6 depicts an illustration of an electrolyte stored in a compartment with a frangible membrane 018. When a system is to be activated, a sharp object 016 can puncture frangible membrane 018, exposing battery cells 014 to electrolyte. Upon activation, controller 005 measures electrical impedance at sensor 017, which will be high in the absence of urine or feces. Controller 005 can continue to measure impedance at sensor 017 over a period of time and can store impedance data in memory 006. Memory 006 can be accessed using receiver 004 from a mobile device or a stationary device to monitor the presence or absence of urine or feces in the diaper of the subject. When urine or feces is excreted by the subject, the impedance will lower relative to the absence, and the admittance will increase relative to the absence, which can prompt controller 005 to transmit all data stored in memory 006 via transmitter 003 to a receiving device (e.g. a mobile device or a stationary device), thereby providing an alert that urine or feces is present.

In some instances, a receiving device can be mounted near a user. For example, a receiving device can be mounted near a hospital bed or wheelchair. Furthermore, a receiving device can be a cloud based system. In some instances, data as described herein can be uploaded to a server via a cloud based system, which can be accessed by a local or remote user for analysis. In some instances, data as described herein can be stored on a cloud based system, and can be retrieved by a user from the cloud based system by an internet connected device.

A controller 005 can be configured to collect impedance and admittance data over a defined or a variable period of time. In some instances, a controller 005 can be programed by a receiving device (e.g. a mobile device or a stationary device) to collect impedance and admittance data over a defined or a variable period of time. In some instances, a controller 005 can collect impedance and admittance data over a defined or a variable period of time by default and without additional programing by a mobile device or a stationary device. In some embodiments, a controller 005 can record a length of waste presence (e.g. urine or feces). For example, a controller 005 can be configured to monitor for the presence of a low-impedance and high-admittance event, such as an initial excretion of urine. Upon reaching a threshold impedance or admittance, a controller 005 can increase the frequency in which impedance and admittance data across the sensor 017 is collected. An increased frequency of impedance and admittance sampling can continue until an admittance value rises above a threshold. Accordingly, by varying the impedance and admittance data sampling rate from low frequency in the absence of waste to high frequency in the presence of waste, a higher density of measurements can be collected in the presence of the waste, while conserving power within the system by limiting the impedance and admittance sampling rate in the absence of waste.

An activated controller 005 can collect impedance and admittance data from sensor 017 at least about every 10, 15, 20, 25, 30, 35, 40, 45, 50, 55, or 60 seconds. In some cases, an activated controller 005 can collect impedance and admittance data from sensor 017 at least about every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 minutes. In some cases, an activated controller 005 can collect impedance and admittance data from sensor 017 at least about every 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, or 24 hours.

Data transmitted by transmitter 003 can be sent to an end user. In some cases, an end user can be a healthcare provider such as a physician, nurse, caregiver, or technician. In some cases, an end user can be a family member or friend of the subject. In some cases, data can be sent to healthcare worker upon initial incidence of urine or feces. If, after a period of time, the urine or feces is still present in the diaper without being changed, the data can be sent to a family member or friend. In some cases, a family member or friend can be notified at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 minutes after excretion of urine or feces.

Also disclosed herein in some embodiments is a method of treating a subject. In some cases, a treatment can be administered to a subject after detection of urine, feces, or a combination thereof. In some cases, a treatment can comprise administering a medicament. In some cases, a medicament can comprise a member selected from the group consisting of: diaper rash cream, zinc oxide, petrolatum, hyaluronic acid, aloe vera, shea butter, jojoba, coconut oil, calendula, vitamin B5, vitamin E, BPH treatment, diarrhea treatment, loperamide, diabetic treatment, insulin, and any combination thereof.

In some cases, a detection of glucose can be used to monitor glucose levels in urine, feces, or a combination thereof. In some cases, a detection of glucose in urine, feces, or a combination thereof can indicate a subject is diabetic. In some cases, a detection of glucose in urine, feces, or a combination thereof can indicate a subject is non-compliant or low-compliant with diabetic medication. In some cases, a detection of an absence of glucose in urine, feces, or a combination thereof can indicate a subject is compliant or substantially compliant with diabetic medication. In some cases, a detection of glucose in urine, feces, or a combination thereof can indicate a subject requires diabetic medication. In some cases, a diabetic medication can comprise insulin. In some cases, monitoring glucose levels can allow evaluation of a course of diabetes treatment. In some cases, a change in treatment strategy can be informed by monitoring glucose levels.

### EXAMPLES

### Example 1—System Construction

A system is constructed comprising an impedance sensor controller by a microcontroller and is embedded in an adult diaper. The impedance sensor contains two parallel elongate sensors embedded in a porous layer of an absorbent pad of a diaper. An RFID transponder and receiver are operatively coupled to the microcontroller for communication with a mobile device. The microcontroller is coupled to onboard RAM for storing impedance data. A battery containing battery cells of copper cathodes and magnesium anodes is operatively coupled to the system. The copper cathodes and magnesium anodes are arranged in cylindrical configuration and are arranged in series. Each battery cell is constructed in an inactive form without electrolyte solution.

A color change stripe is included in the absorbent pad. The stripe is configured to change color when contacted with moisture (e.g. urine or feces).

### Example 2—Alternative System Construction

A system is constructed comprising an impedance sensor controller by a microcontroller and is embedded in an adult diaper. The impedance sensor contains two parallel elongate sensors embedded in a porous layer of an absorbent pad of a diaper. An RFID transponder and receiver are operatively coupled to the microcontroller for communication with a mobile device. The microcontroller is coupled to onboard RAM for storing impedance and admittance data. A battery containing battery cells of copper cathodes and magnesium anodes is operatively coupled to the system. The copper cathodes and magnesium anodes are arranged in cylindrical configuration and are arranged in series. Each battery cell is constructed in an inactive form without electrolyte solution.

A chamber storing potassium chloride electrolyte solution is placed adjacent to the battery cells and is separated from the battery cells by a frangible membrane. A sharp tack is placed adjacent the frangible membrane, which will rupture the frangible membrane when pressed, thereby contacting the electrolyte solution with the battery cells.

An audible speaker unit is operatively coupled to the microcontroller. The microcontroller is programmed to produce an audible noise via the audible speaker unit when the impedance drops at least approximately 20% from a baseline impedance, or when the admittance increases at least approximately 20% from a baseline admittance, that is collected when the system is activated by rupturing of the frangible membrane.

### Example 3—Activation by Urine

A subject in a nursing home is fitted with a diaper constructed as described in Example 1. The diaper is in an inactive state in the absence of urine due to the sequestration of the electrolyte solution from the battery cells. Upon excretion of urine by the subject, the urine coats the absorbent pad and makes contact with the battery cells and the elongate electrodes of the sensor. Upon contacting the sensor, the urine allows for electrical charge transfer between the anode and cathode, thus activating the battery. Once activated, the battery provides power to the controller, which begins collecting impedance and admittance data at the sensor along the elongate electrodes. The impedance is calculated by dividing the applied voltage by the resistance. The admittance is calculated by dividing the impedance into 1. Where urine is present, the impedance is lowered relative to the absence of urine, and the admittance is increased relative to the absence of urine. Once urine is detected, the microcontroller transmits RF signals to a mobile device or stationary device. Based on the duration and/or frequency of the urine detection signal, the mobile device or stationary device may send an alert to nurses at a nursing station that urine is present and the diaper needs to be changed due to possible saturation. After 15 minutes has elapsed, a second alert is sent to the nurses that urine is present and the diaper needs to be changed. After 15 minutes has further elapsed, an alert is sent to a family member via a push notification to the family member's phone alerting the family member that urine saturation may have been present in the diaper for 30 minutes.

### Example 4—Activation by Feces

A subject in a hospital is fitted with a diaper constructed as described in Example 1. The diaper is in an inactive state in the absence of feces due to the sequestration of the electrolyte solution from the battery cells. Upon excretion of feces by the subject, the feces contacts the absorbent pad and contacts the battery cells and the elongate electrodes of the sensor. Upon contacting the sensor, the faces allows for electrical charge transfer between the anode and cathode, thus activating the battery. Once activated, the battery provides power to the controller, which begins collecting impedance and admittance data at the sensor along the elongate electrodes. The impedance is calculated by dividing the applied voltage by the resistance. The admittance is calculated by dividing the impedance into 1. Where feces is present, the impedance is persistent and low relative to the absence of feces. Where feces is present, the admittance is high and persistent relative to the absence of feces. Once feces is detected, the microcontroller immediately transmits an alert, via a mobile device or stationary device, to nurses at a nursing station that feces is present. After 15 minutes has elapsed, a second alert is sent to the nurses that feces is present. After 15 minutes has further elapsed, an alert is sent to a family member via a push notification to the family member's phone alerting the family member that feces has been present in the diaper for 30 minutes.

### Example 5—Monitoring of Incontinence in a Clinical Trial

A subject enrolled in a clinical trial for incontinence is fitted with a diaper constructed as described in Example 2. The diaper is in an inactive state in the absence of urine due to the sequestration of the electrolyte solution from the battery cells. Prior to the beginning of the clinical trial, the tack is depressed, which ruptures the frangible membrane and activates the battery and system.

The excretion of urine is monitored over time in the clinical trial by measuring the impedance and admittance over time. The microcontroller is programmed to record impedance and admittance measurements every 5 minutes after administration of treatment for incontinence. In the absence of urine, the impedance between the elongate electrodes is high, and the admittance is low. Upon excretion of urine by the subject, the urine coats the absorbent pad and makes contact with the elongate electrodes of the sensor. The initial contact of the urine with the sensor produces a transient decrease in impedance, and a transient increase in admittance. After absorption of the urine into the absorbent pad, the impedance gradually increases, and the admittance gradually decreases. The impedance and admittance data stored in the memory is accessed by clinical trial personnel to monitor the progression of the treatment via the RFID transponder/receiver.

### Example 6—Monitoring of Urine

A subject in a hospital is fitted with a diaper constructed as described in Example 2. The diaper is in an inactive state in the absence of urine due to the sequestration of the electrolyte solution from the battery cells. Upon fitting of the diaper, the tack is depressed, which ruptures the frangible membrane and activates the battery and system.

Using a mobile device paired with the diaper via an RFID connection, a healthcare worker can program the microcontroller of the diaper to record impedance and admittance measurements every 15 minutes. The excretion of urine is monitored over time in the hospital by measuring the impedance and admittance over time. In the absence of urine, the impedance between the elongate electrodes is high, and the admittance is low. Upon excretion of urine by the subject, the urine coats the absorbent pad and makes contact with the elongate electrodes of the sensor. The initial contact of the urine with the sensor produces a transient decrease in impedance and a transient increase in admittance. After absorption of the urine into the absorbent pad, the impedance gradually increases, and the admittance gradually decreases. The impedance and admittance data stored in the memory is accessed by the hospital worker using the mobile device paired with the system. Upon retrieving the data from the system, the hospital worker performs post processing on the data to plot the frequency of urination over time. The post processing analysis links the data plot to the subject's personal profile.

### Example 7—Monitoring of Feces

A subject in a hospital is fitted with a diaper constructed as described in Example 2. The diaper is in an inactive state in the absence of feces due to the sequestration of the electrolyte solution from the battery cells. Upon fitting of the diaper, the tack is depressed, which ruptures the frangible membrane and activates the battery and system.

Using a mobile device paired with the diaper via an RFID connection, a healthcare worker can program the microcontroller of the diaper to record impedance and admittance measurements every 15 minutes. The excretion of feces is monitored over time in the hospital by measuring the impedance and admittance over time. In the absence of feces, the impedance between the elongate electrodes is high, and the admittance between the elongate electrodes is low. Upon excretion of feces by the subject, the impedance is persistent and low relative to the absence of feces, and the admittance is persistent and high relative to the absence of feces. The impedance and admittance data stored in the memory is accessed by the hospital worker using the mobile device paired with the system. Upon retrieving the data from the system, the hospital worker performs post processing on the data to plot the frequency of defecation over time. The post processing analysis links the data plot to the subject's personal profile.

While exemplary embodiments have been shown and described herein, such embodiments are by way of example only. Numerous variations, changes, and substitutions can be performed on the exemplary embodiments. It should be understood that various alternatives to the embodiments described herein may be employed.

What is claimed is:

1. A system comprising:
(a) a processor;
(b) a battery comprising an anode and a cathode, and
(c) at least one sensor comprising a working electrode and a counter electrode, wherein the at least one sensor is operatively coupled to the battery and the processor;
wherein the processor is configured to measure an impedance, across the working electrode and the counter electrode of the at least one sensor, in response to feces or urine, thereby generating data that is indicative of urine, feces, or a combination thereof; and
wherein an electrical circuit is substantially open in the absence of urine, feces, or an electrolyte solution, and is at least partially closed when the sensor is contacted with urine, feces, or an electrolyte solution; thereby providing power to the processor and sensor only in the presence of urine, feces, or the electrolyte solution.

2. The system of claim 1, further comprising a radio-frequency identification (RFID) transponder operatively coupled to the processor and the battery.

3. The system of claim 2, wherein the RFID transponder is configured to transmit the data in the presence of urine, feces or the electrolyte solution.

4. The system of any one of claims 1-3, wherein the system does not measure a threshold level of current in the absence of urine, feces or electrolyte solution.

5. The system of claim 4, wherein the urine, the feces, the electrolyte solution or a combination thereof produces the threshold level of impedance.

6. The system of any one of claim 1-4, wherein the data is impedance data.

7. The system of any one of claims 1-6, further comprising a memory module operatively coupled to the processor and the battery, wherein the memory module is configured to store the data.

8. The system of claim 7, wherein the RFID transponder is configured to access the memory module.

9. The system of any one of claims 1-8, further comprising an RFID receiver operatively coupled to the processor and the battery.

10. The system of claim 9, wherein the RFID receiver is configured to receive instructions from a device by an RF signal to transmit the data to the device.

11. The system of claim 10, wherein the RFID transponder is configured to transmit the data to the device by an RF signal.

12. The system of claim 10 or 11, wherein the device is a mobile device or a stationary device.

13. The system of claim 10 or 11, wherein the device is mounted close to a user.

14. The system of claim 6, wherein the generating of the data comprises an analog to digital conversion of voltage.

15. The system of any one of claims 1-14, comprising a plurality of sensors.

16. The system of claim 15, wherein the plurality of sensors are arranged in at least two layers on a surface.

17. The system of any one of claims 1-16, wherein the sensor comprises a glucose sensor.

18. A diaper comprising the system of any one of claims 1-17 and an absorbent pad.

19. The diaper of claim 18, further comprising a reservoir storing the electrolyte solution.

20. The diaper of claim 19, wherein the reservoir comprises a frangible membrane, wherein the electrolyte solution is at least partially sequestered from the battery by the frangible membrane, and wherein the electrolyte solution contacts the battery when the frangible membrane is ruptured.

21. The diaper of claim 18, further comprising a transmitter configured to transmit the impedance data to a device.

22. The diaper of claim 21, wherein the battery comprises magnesium or a salt thereof.

23. The diaper of claim 22, wherein the battery comprises from about 10% to about 90% by weight of the magnesium or salt thereof with respect to a total weight of the battery.

24. The diaper of claim 21, wherein the battery comprises copper or a salt thereof.

25. The diaper of claim 24, wherein the battery comprises from about 10% to about 90% by weight of the copper or salt thereof with respect to a total weight of the battery.

26. The diaper of claim 19, further comprising an object configured to pierce the frangible membrane.

27. The diaper of any one of claims 18-26, wherein the absorbent pad comprises a plurality of sensors.

28. The diaper of claim 27, wherein the plurality of sensors are arranged in at least two layers on the absorbent pad.

29. The diaper of claim 28, further comprising a porous or hydrophobic material layer at least partially covering the plurality of sensors.

30. The diaper or any one of claims 18-29, further comprising a visual indicator configured to provide a visual prompt in the presence of urine, feces, or a combination thereof.

31. The diaper or any one of claims 18-29, further comprising an audio indicator configured to provide an audio prompt in the presence of urine, feces, or a combination thereof.

32. The diaper of any one of claims 18-29, further comprising a processor operatively coupled to the at least one sensor and the battery.

33. The diaper of claim 32, wherein the processor is configured to measure an impedance across the working electrode and the counter electrode of the at least one sensor in response to the feces, the urine, or the electrolyte solution that is in contact with the absorbent pad, thereby generating impedance data that is indicative of the urine or the feces.

34. The diaper of any one of claims 18-33 that is sized to fit a human.

35. The diaper of claim 34, wherein the human is from about 0 years old to about 17 years old.

36. The diaper of claim 35, wherein the human is from about 0 years old to about 4 years old.

37. The diaper of claim 34, wherein the human is from about 18 years old to about 120 years old.

38. The diaper of claim 37, wherein the human is from about 65 years old to about 120 years old.

39. A method comprising:
(a) detecting feces or urine by contacting the feces or urine with the at least one sensor of the system of any one of claims 2-17; thereby generating the data; and
(b) transmitting the data to a device using the RFID transponder.

40. A method comprising:
(a) detecting feces or urine by contacting the feces or urine with the absorbent pad of the diaper of any one of claims 18-38; thereby generating the data; and
(b) transmitting the data to a device using the transmitter.

41. The method of claim 39 or 40, wherein the data is stored on a mobile device or a stationary device, displayed on the mobile device or the stationary device, or any combination thereof.

42. The method of claim 39 or 40, wherein the data is at least partially uploaded to a remote server via a cloud based system.

43. The method of claim 41, wherein an analysis of the data is at least partially performed on the mobile device or the stationary device.

44. The method of claim 42, wherein an analysis of the data is performed at least partially on the remote server.

45. The method of claim 43 or 44, wherein the analysis comprises an alert of the presence of urine or feces.

46. The method of claim 43 or 44, wherein the analysis comprises an alert of urine saturation based on a duration the urine or a frequency of the data.

47. The method of claim 43, wherein the analysis comprises measuring the presence of urine or feces as a function of time.

48. The method of claim 40, further comprising rupturing the frangible membrane.

49. The method of claim 48, further comprising contacting the battery with the electrolyte solution.

50. The method of any one of claims 39-47, wherein the detecting is performed on a human.

51. The method of claim 50, wherein the human is age about 0-17 years old.

52. The method of claim 50, wherein the human is age about 18-120 years old.

53. The method of any one of claims 39-52, wherein the sensor comprises a glucose sensor, and wherein the method further comprises detecting a level of glucose in the urine, feces, or combination thereof by contacting urine, feces, or a combination thereof with the glucose sensor.

54. The method of claim 53, further comprising monitoring the level of glucose in the urine, feces, or combination thereof over a period of time.

55. The method of claim 54, further comprising determining compliance with a diabetic treatment based on the level of glucose in a subject administered the diabetic treatment.

56. The method of any one of claims 50-55, further comprising administering a medicament to the human.

57. The method of claim 56, wherein the medicament is selected from the group consisting of: diaper rash cream, zinc oxide, petrolatum, hyaluronic acid, aloe vera, shea butter, jojoba, coconut oil, calendula, vitamin B5, vitamin E, BPH treatment, diarrhea treatment, loperamide, diabetic treatment, insulin, and any combination thereof.
